(19) United States
(12) Patent Application Publication
(10) Pub. No.: US 2014/0135651 A1
(43) Pub. Date: May 15, 2014

(54) Urine Specimen Collection Device

(71) Applicant: Jobsono Pty Ltd ATF The Leeke Family Super Fund, Byron Bay (AU)

(72) Inventors: Wallace LEEKE, Byron Bay (AU); Wendy Leeke, Byron Bay (AU); Ric B. Richardson, Ewingsdale (AU)

(73) Assignee: Jobsono Pty Ltd ATF The Leeke Family Super Fund, Byron Bay (AU)

(21) Appl. No.: 14/078,555

(22) Filed: Nov. 13, 2013

(30) Foreign Application Priority Data
Nov. 14, 2012 (AU) 2012904956
Jan. 31, 2013 (AU) 2013100088

(51) Int. Cl. A61B 10/00

(52) U.S. Cl. CPC A61B 10/007
USPC 600/574

(57) ABSTRACT

A urine collection device for human females; said collection device comprising a funnel-shaped upper portion extending to a cylindrical connecting collar; said collar provided with inwardly projecting engagement structures; said structures adapted to engage with at least two different thread configurations of externally threaded upper portions of urine specimen collection jars; said collar further including an internal flange for sealing engagement with an upper rim of a said urine specimen collection jar.

### TECHNICAL FIELD

The present invention relates to urine specimen collection and, more particularly to devices adapted for collection of urine specimens from human females.

### BACKGROUND

Urine specimen collection jars are known in the art. One drawback of current designs is that they do not allow the easy collection of urine especially for women who are generally unable to view the jar during collection and must approximate the position of the jar for collection. This approach leads to spillage, overfilling and possible contamination of the specimen.

Various funnels to help guide urine into the specimen jar during collection are known in the art, however the disclosed arrangements often require expensive and complicated components and manufacturing processes that make the designs less desirous in a price conscious market.

Urine specimen collection devices for connection to urine specimen collection jars are also known in the art. For example U.S. Pat. No. 6,299,606 discloses a funnel-like device provided with an internally threaded collar for attachment of a specimen collection jar.

A disadvantage of the device of U.S. Pat. No. 6,399,606 and similar arrangements is that the threaded connection is realised in a standard continuous helical thread specific to a particular thread specification. Specimen jars feature a water tight, internally threaded lid. Despite the similarities of jar design and diameters, different manufacturers use different thread specifications so that a collecting funnel suitable for the jar of one manufacturer cannot accommodate the jar from another.

It is an object of the present invention to address or at least ameliorate some of the above disadvantages or provide a useful alternative.

### NOTES

The term “comprising” (and grammatical variations thereof) is used in this specification in the inclusive sense of “having” or “including”, and not in the exclusive sense of “consisting only of”.

The above discussion of the prior art in the Background of the invention, is not an admission that any information discussed therein is citable prior art or part of the common general knowledge of persons skilled in the art in any country.

### SUMMARY OF INVENTION

Accordingly in one broad form of the invention there is provided a urine collection device for human females; said collection device comprising a funnel-shaped upper portion extending to a cylindrical connecting collar; said collar provided with means for accommodative engagement adapted to sealingly engage with at least two different thread configurations of externally threaded upper portions of urine specimen collection jars.

Preferably the internal diameter of the flange is less than the internal diameter of the neck portion of the collection jar whereby, in use, the liquid stream is directed centrally within the neck portion of the collection jar and clear of the seal between the urine collection device 10 and the collection jar or device.

Preferably the means for accommodative engagement includes inwardly extending projections.

Preferably said funnel-shaped upper portion is shaped to fit about the vulva of a said human female; said upper portion having a generally elongate opening.

Preferably said at least two different thread configurations of said urine specimen collection jars include a relatively coarse pitched thread and a relatively fine pitched thread.

Preferably said inwardly extending projections of said cylindrical connecting collar are flexible projections arranged in a pattern; at least a proportion of said flexible projections lying along respective paths defined by said relatively coarse pitched thread and said relatively fine pitched thread when a said specimen collection jar is assembled to said urine collection device.

Preferably upper surfaces of at least a proportion of said projections engage with an underside of a said relatively coarse pitched thread of a relatively fine pitched thread so as to support said thread.

Preferably said flexible projections are not aligned with a said path of a thread of a said specimen collection jar are deformed sufficiently to not impede attachment of a said specimen collection jar to said urine collection device.

Preferably an upper rim of a said specimen collection jar forms a substantially liquid proof seal with an internal flange of said urine collection device when said specimen collection jar is fully engaged within said connecting collar of said urine collection device.

Preferably the internal flange diameter is less than or equal to the diameter of the specimen collection jar.

In yet a further broad form of the invention there is provided a method of connecting a urine collection device to specimen collection jars; and wherein each jar can have a different thread connection one from the other; said method including the step of:

(a) preparing an internal surface of a connecting collar of said urine connection device with means for accommodative engagement with a respective one of said different thread connection.

Preferably the means for accommodative engagement comprises flexible inwardly extending projections.

Preferably the method further includes arranging said flexible inwardly extending projections on said internal surface such that at least a proportion of said projections lie on paths defined by said at least two different thread specifications when respective specimen collection jars with said different thread specifications are fully engaged in said connecting collar.

Preferably upper surfaces of said at least a proportion of said flexible inwardly extending projections engage with undersides of a thread of either of said at least two different thread specifications so as to support said specimen collection jar in said connecting collar.

Preferably flexibility of said flexible inwardly extending projections not aligned with a said thread of a said specimen collection jar is such as to not impede engagement of said specimen collection jar with said connecting collar.

### BRIEF DESCRIPTION OF DRAWINGS

Embodiments of the present invention will now be described with reference to the accompanying drawings wherein:

FIG. 1 is a cross section view of a urine collection device according to a preferred embodiment of the invention and a separate urine specimen collection jar,

FIG. 2 is a cross section view of the urine collection device and urine specimen collection jar of FIG. 1 assembled together for use,

FIG. 3 is an enlarged cross section view of a portion of a collar at a lower portion of the urine collection device of FIGS. 1 and 2, showing engagement between the external thread of a urine specimen collection jar and engagement elements within the collar according to the invention,

FIG. 4 is a general view from above of the urine collection device of FIGS. 1 to 3 showing the elongate opening of the funnel-shaped upper portion,

FIGS. 5A and 5B show two thread pattern examples at the periphery of typical specimen jars externally threaded collars,

FIG. 5C is a representation of the internal surface of the collar of FIG. 3 showing a preferred embodiment of the engagement elements according to the invention,

FIG. 6 is an enlarged cross section view of an element of the thread of a specimen collection jar and an inwardly projecting engagement element of the collar of the urine collection device of the invention, in one form of engagement,

FIG. 7 is a further enlarged cross section view of the thread of a specimen collection jar and the urine collection device of the invention in which a projecting engagement element is deflected in a second form of engagement.

FIG. 8 is a view of a further embodiment of flexible inwardly extending projections provided in a connecting collar of the invention.

### DESCRIPTION OF EMBODIMENTS

With reference to FIGS. 1, 2 and 4, the urine sample collection device 10 according to the invention, comprises a funnel-shaped upper portion 12 extending to a cylindrical connecting collar 14. The upper opening 16 of the funnel-shaped upper portion 12 is preferably of an elongate narrow shape to more comfortably fit against the vulva of a human female (not shown).

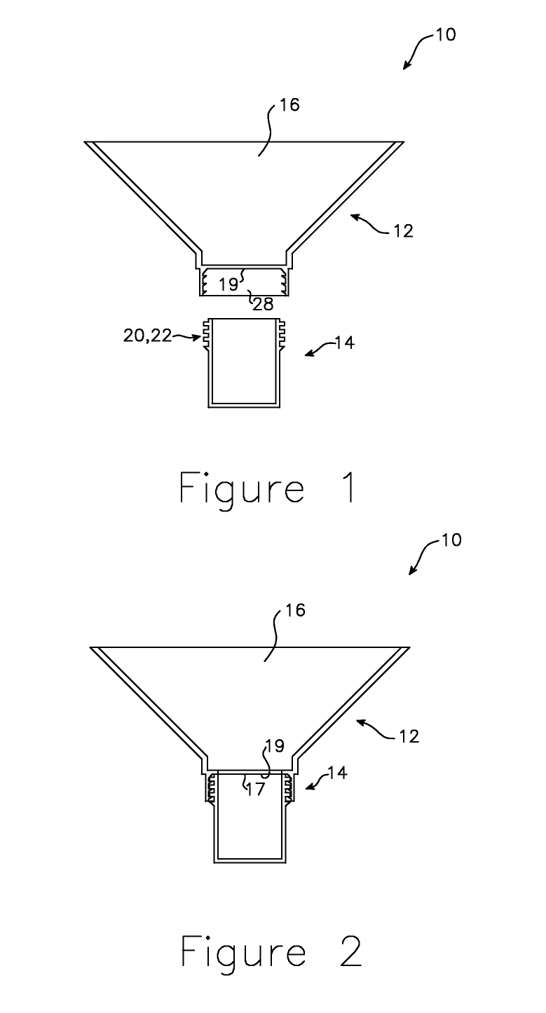

The cylindrical connecting collar 14 of the specimen collection device 10 has an internal diameter adapted to accept the thread of a specimen collection jar 18 so that when assembled together as shown in FIG. 2, the upper rim 17 of the specimen collection jar 18 forms a substantially liquid tight seal against an internal flange 19 provided at the junction between the funnel-shaped upper portion 12 and the cylindrical connecting collar 14.

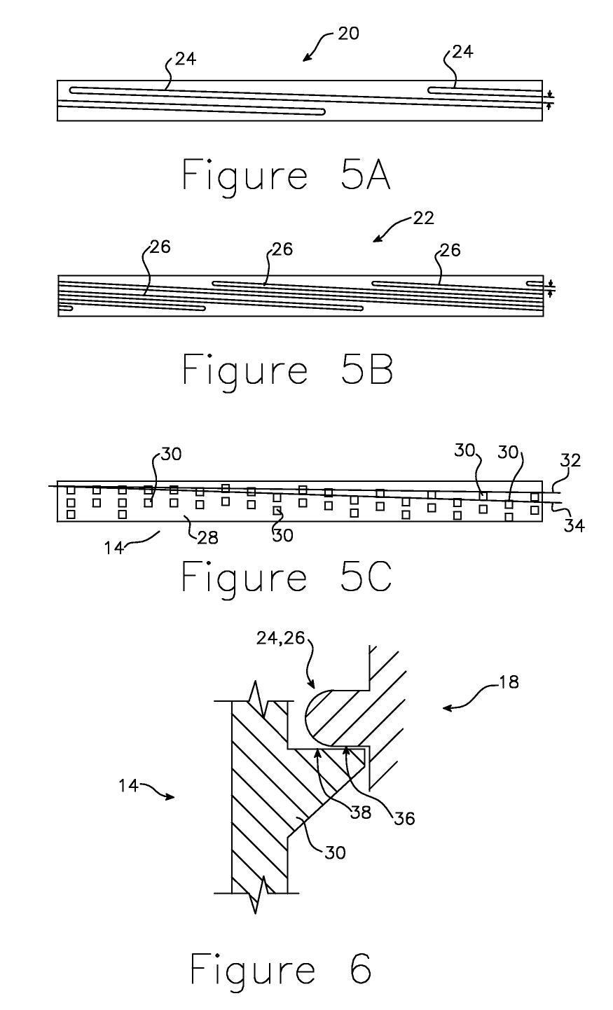

The urine collection device 10 of the invention is adapted for attachment to specimen collection jars with at least two different thread specifications, though with similar root diameters, as known in the art. As shown in FIGS. 5A and 5B, which represent the developed external surfaces of the threaded upper portions 20 and 22 respectively of two specimen jars, the cylindrical connecting collar 14 is adapted to engage with the relatively wide, coarse pitched thread 24 of FIG. 5A and the relatively narrow, fine pitched thread 26. of FIG. 5B.

A particular feature of the specimen collection device of the present invention is that the internal surface 28 of the cylindrical connecting collar 14 (shown developed in FIG. 5C) is not provided with a continuous helical thread as is the case for example in the prior art device of U.S. Pat. No. 6,299,606 discussed above. Instead the internal surface 28 of the collar 14 of the present device 10 is provided with an array of flexible projections 30 as shown in FIG. 5C.

This pattern of flexible projections 30 is arranged so that at least a proportion of the flexible projections 30 are located along respective paths 32 and 34 as defined by the relatively coarse pitched thread 24 and the relatively fine pitched thread 26 when a specimen collection jar is assembled to the specimen collection device 10. Thus the pattern is arranged in such a way as to provide maximum engagement between the projections 30 and the helical threads 24 and 26 of a specimen collection jar.

FIG. 6 shows how one projection 30 engages with a thread portion 24 or 26 of a specimen collection jar 18. In this case the thread portion 24 or 26 at this particular location of the projection 30 within the cylindrical connecting collar 14 engages with the projection 30 so that the underside 36 of the thread is supported on the upper surface 38 of the projection 30.

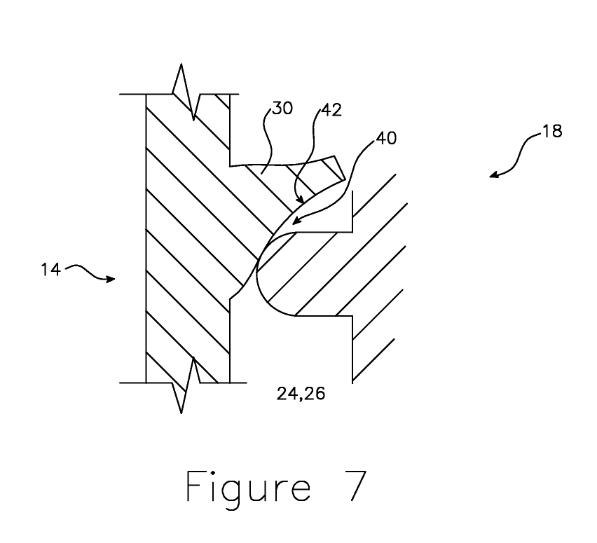

FIG. 7 shows another flexible projection 30 when being deformed by a thread element 24 or 26. This situation may occur at those projections where it is not possible to accommodate completely the particular helical thread path. As the specimen collection jar 18 is engaged with the cylindrical connecting collar 14, sufficient of the projections 30 will engage with the underside of the threads 24 or 26 as shown in FIG. 16 to secure the specimen collection jar 18 within the collar 14.

Those of the projections 30 not ideally placed to engage with the collecting jar thread without snagging (as shown in FIG. 6), will start to deform and yield to the increasing upward pressure applied by the rising thread as the specimen collection jar 18 rotates relative the urine collection device 10 during engagement. The flexibility of the projections is such that the deformation is sufficient not to impede the process of attaching a specimen collection jar to the collection device of the invention.

It is an intentional design feature of the urine specimen collection device 10 of the invention to allow, by the selection of suitable flexible material, the deformation of those projections 30 which may be misaligned with a specimen jar thread.

Although the deformation of some of the projections may not be permanent, it is anticipated that the device should be single use only since the funnel-like upper portion 12 will be contaminated and unsuitable for reuse.

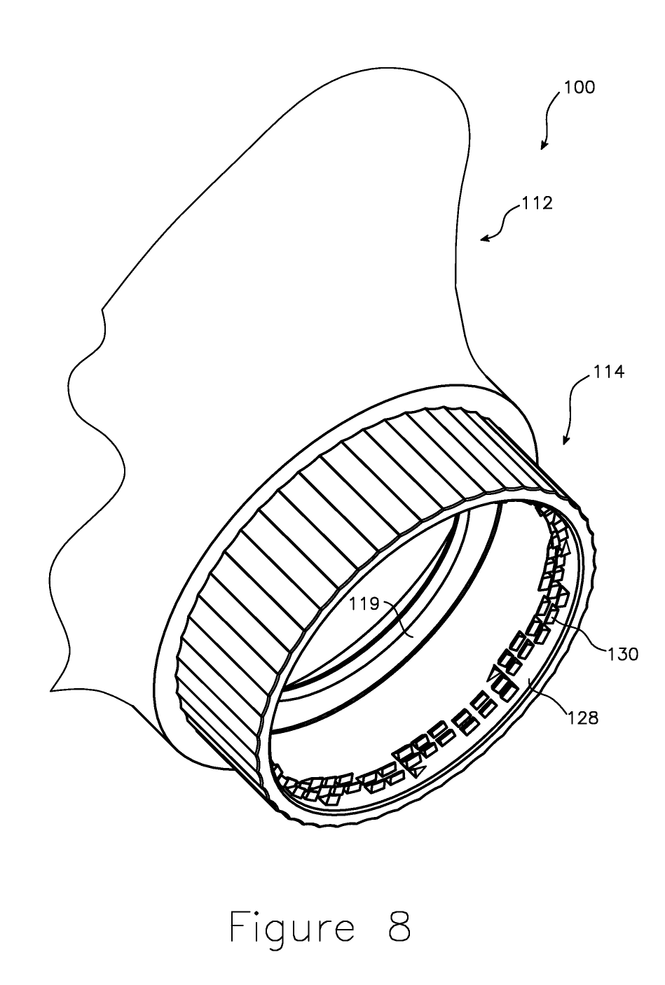

FIG. 8 shows a further embodiment of a cylindrical connecting collar 114 of the urine specimen device 100 according to the invention. In this embodiment, the flexible inwardly extending projections 130 are formed on the internal surface 128 of the collar 114 as a discontinuous thread formation.

### INDUSTRIAL APPLICABILITY

The present invention provides a more convenient and hygienic device and method for the collection of a urine specimen in a standard specimen jar. In particular, the collection device of the invention allows for engagement with at least two different types of thread by means of the unique projecting elements within the cylindrical connecting collar of the device.

In use, the closing lid of the specimen jar is removed and the jar screwed into the connecting collar of the collection device, with a normal screwing action. After collection the specimen jar is removed from the device and the lid replaced.

### SUMMARY

FIG. 1 discloses the example embodiment 10 in a separate unattached state to the specimen collection jar 18. Both 10 and 18 are viewed in cross-section. The example embodiment includes an attachment capability that attaches to the screw thread of the specimen jar 18.

The example embodiment 10 also includes a funnel shape 12 that allows easy and comfortable collection of urine by the user. The urine is collected on the inside of the funnel shape and directed to the specimen jar 18 for collection.

FIG. 2 shows the example embodiment 10 in an attached state with the specimen jar 18. In this view the attachment capability and the screw thread of the specimen jar are engaged.

FIG. 4 shows a plan view of the example embodiment in plan view where the top of the funnel shape 12 allows a slope down to a central outlet that is less than or equal to the top of the specimen jar 18 in circumference.

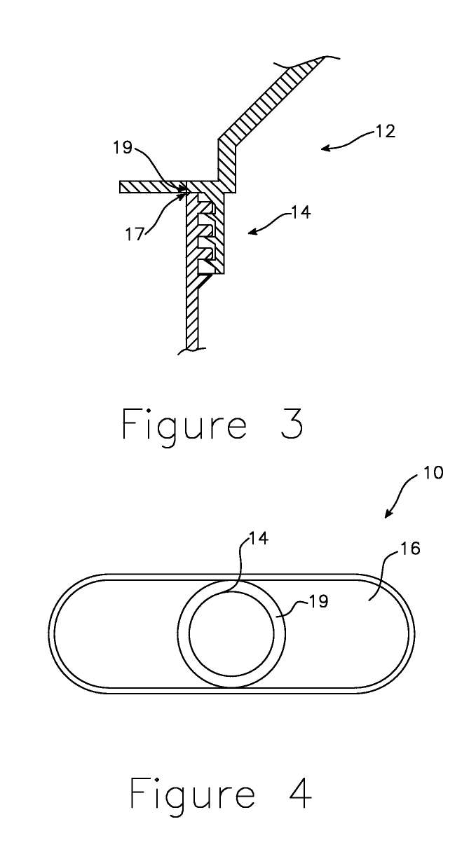

FIG. 3 discloses an expanded view of the attachment system between the example embodiment 10 and the specimen jar 18. The threads 24, 26 of the specimen jar are engaged against the top side of the angled prongs or projections 30 of the example embodiment so that a secure attachment is made. Ideally the top of the specimen jar 18 and the underside of the example embodiment funnel 12 are sealed to a watertight or near watertight degree.

In a particularly preferred form (suited to the situation where the seal is perhaps not fully liquid-tight) the internal flange 19 is sized and shaped to overhang the seal. With reference to FIGS. 3 and 4 in a preferred form this is achieved by the internal diameter of the flange 19 being less than the internal diameter of the neck portion of the collection jar whereby , in use, the liquid stream is directed centrally within the neck portion of the collection jar and clear of the seal between the urine collection device 10 and the collection jar or device 18.

Multiple spaced prongs or projections 30 are used to allow maximum attachment between the example embodiment 10 and the specimen jar 18.

FIGS. 5A and 5B disclose two examples 20 and 22 of different types of threads used on different brands of urine collection jars known in the art. One example 20 uses a wide thread 24 with a coarse pitch. The second example 22 uses a smaller thread 26 with a finer pitch.

The example embodiment 10 uses a measured placement of prongs or projections 30 that are placed along both the path of the coarse pitch thread 24 and the fine pitch thread 26 in such a way so as to allow maximum engagement between the attachment prongs or projections 30 and the threads of either the fine thread 24 or the coarse thread 26.

FIG. 6 discloses a prong or projection from the example embodiment 10 engaging with the thread 24 or 26 of a specimen jar 18. The top side 38 of the projection 30 engages with the underside 36 of the specimen jar thread.

FIG. 7 discloses a projection 30 of the example embodiment in the process of being deformed during attachment with a specimen jar 18. This situation may occur where it is not possible to accommodate completely the thread paths of two different thread pitches or thread sizes. During attachment, the prongs engage the underside of the thread of the specimen jar but some prongs are not ideally placed to engage the jar screw without snagging. In this case the top side 40 of a jar screw 24 or 26 starts to engage with the underside 42 of a projection 30 positioned on the inside of the example embodiment 10. As more pressure is applied, the material of the example embodiment 10 which is usually plastic will start to deform and accommodate the upward pressure of the jar screw thread 24 or 26.

It is an intentional design feature of the example embodiment to allow deformation of the attachment prongs 94 to allow secure attachment of the example embodiment with the specimen jar.

The deformation of the projection 30 of the example embodiment 10 during use is anticipated and within the design criteria of the operation of the device on the basis that the reuse of the funnel after sample collection is not desired since the funnel is now contaminated and not available for sterile reuse in the specimen collection process.

The above describes only some embodiments of the present invention and modifications, obvious to those skilled in the art, can be made thereto without departing from the scope of the present invention.

1. A urine collection device for human females; said collection device comprising a funnel-shaped upper portion extending to a cylindrical connecting collar; said collar provided with inwardly projecting engagement structures; said structures adapted to engage with at least two different thread configurations of externally threaded upper portions of urine specimen collection jars or like devices; said collar further including an internal flange for sealing engagement with an upper rim of a said urine specimen collection jar or device.

2. The collection device of claim 1 wherein the internal diameter of the flange is less than the internal diameter of the neck portion of the collection jar whereby , in use, the liquid stream is directed centrally within the neck portion of the collection jar and clear of the seal between the urine collection device and the collection jar or device.

3. The urine collection device of claim 1 wherein said funnel-shaped upper portion is shaped to fit about the vulva of a said human female; said upper portion having a generally elongate opening.

4. The urine collection device of claim 1 wherein said inwardly projecting engagement structures of said cylindrical connecting collar are flexible projections arranged in a pattern to engage with a relatively coarse pitched thread and a relatively fine pitched thread of said urine specimen collection jars; at least a proportion of said flexible projections lying along respective paths defined by said relatively coarse pitched thread and said relatively fine pitched thread when a said specimen collection jar is assembled to said urine collection device.

5. The urine collection device of claim 4 wherein upper surfaces of at least a proportion of said projections engage with an underside of a said relatively coarse pitched thread or a relatively fine pitched thread so as to support a respective said urine specimen collection jar within said cylindrical connecting collar; said flexible projections not aligned with a said path of a thread of a said specimen collection jar are deformed sufficiently to not impede attachment of a said specimen collection jar to said urine collection device.

6. A method of connecting a urine collection device to any one of at least two specimen collection jars where each jar is provided with different thread specifications; said method including the step of:
(a) preparing an internal surface of a cylindrical connecting collar of said urine connection device with flexible inwardly extending projections for engagement with a respective one of said different thread connections,
(b) arranging said flexible inwardly extending projections on said internal surface such that at least a proportion of said projections lie on paths defined by said different thread specifications when respective specimen collection jars with said different thread specifications are fully engaged in said connecting collar, and
wherein flexibility of said flexible inwardly extending projections is such that any of said flexible inwardly extending projections not aligned with a said thread of a said specimen collection jar does not impede engagement of said specimen collection jar with said connecting collar.

7. A urine collection device for human females; said collection device comprising a funnel-shaped upper portion extending to a cylindrical connecting collar;
said collar provided with means for accommodative engagement adapted to sealingly engage with at least two different thread configurations of externally threaded upper portions of urine specimen collection jars.

8. The urine collection device of claim 7 wherein the means for accommodative engagement includes inwardly extending projections.

9. The urine collection device of claim 7 wherein said funnel-shaped upper portion is shaped to fit about the vulva of a said human female; said upper portion having a generally elongate opening.

10. The urine collection device of claim 1 wherein said at least two different thread configurations of said urine specimen collection jars include a relatively coarse pitched thread and a relatively fine pitched thread.

11. The urine collection device of claim 7 wherein said inwardly extending projections of said cylindrical connecting collar are flexible projections arranged in a pattern; at least a proportion of said flexible projections lying along respective paths defined by said relatively coarse pitched thread and said relatively fine pitched thread when a said specimen collection jar is assembled to said urine collection device.

12. The urine collection device of claim 11 wherein upper surfaces of at least a proportion of said projections engage with an underside of a said relatively coarse pitched thread of a relatively fine pitched thread so as to support said thread.

13. The urine collection device of claim 11 wherein said flexible projections not aligned with a said path of a thread of a said specimen collection jar are deformed sufficiently to not impede attachment of a said specimen collection jar to said urine collection device.

14. The urine collection device of claim 7 wherein an upper rim of a said specimen collection jar forms a substantially liquid proof seal with an internal flange of said urine collection device when said specimen collection device is fully engaged within said connecting collar of said urine collection device.

15. A method of connecting a urine collection device to specimen collection jars; and wherein each jar can have a different thread connection one from the other; said method including the step of:
(a) preparing an internal surface of a connecting collar of said urine connection device with means for accommodative engagement with a respective one of said different thread connection.

16. The method of claim 15 wherein the means for accommodative engagement comprises flexible inwardly extending projections.

17. The method of claim 16 further including arranging said flexible inwardly extending projections on said internal surface such that at least a proportion of said projections lie on paths defined by said at least two different thread specifications when respective specimen collection jars with said different thread specifications are fully engaged in said connecting collar.

18. The method of claim 17 wherein upper surfaces of said at least a proportion of said flexible inwardly extending projections engage with undersides of a thread of either of said at least two different thread specifications so as to support said specimen collection jar in said connecting collar.

19. The method of claim 16 wherein flexibility of said flexible inwardly extending projections not aligned with a said thread of a said specimen collection jar is such as to not impede engagement of said specimen collection jar with said connecting collar.

20. The urine collection device of claim 7 wherein said at least two different thread configurations of said urine specimen collection jars include a relatively coarse pitched thread and a relatively fine pitched thread.
